(19) United States
(12) Patent Application Publication
(10) Pub. No.: US 2013/0225819 A1
(43) Pub. Date: Aug. 29, 2013

(54) USE OF AZIDES IN SYNTHESIS

(71) Applicants: Timothy F. Jamison, Somerville, MA (US); Prakash B. Palde, Somerville, MA (US)

(72) Inventors: Timothy F. Jamison, Somerville, MA (US); Prakash B. Palde, Somerville, MA (US)

(73) Assignee: Massachusetts Institute of Technology, Cambridge, MA (US)

(21) Appl. No.: 13/817,291

(22) PCT Filed: Aug. 18, 2011
(86) PCT No.: PCT/US11/48261

§ 371 Date: May 8, 2013

Related U.S. Application Data
(60) Provisional application No. 61/375,020, filed on Aug. 18, 2010.

(51) Int. Cl. C07D 257/04; C07D 401/04; C07D 403/04

(52) U.S. Cl. CPC C07D 257/04; C07D 403/04; C07D 401/04
USPC 544/405; 548/250; 548/252; 548/253; 546/210; 546/167

(57) ABSTRACT

Described herein are inventive methods for synthesis of tetrazoles. In some embodiments, the method involves the use of a flow reactor. The methods provided herein are capable at being carried out in short reaction times, with high yields, with minimal side reactions, and/or with minimal chance of explosions caused by the presence of azides.

### RELATED APPLICATIONS

The present application claims priority under 35 U.S.C. §119(e) to U.S. provisional application, U.S. Ser. No. 61/375,020, filed Aug. 18, 2010, entitled “Use of Azides in Synthesis,” incorporated herein by reference in its entirety for all purposes.

### FIELD OF INVENTION

Described herein are inventive methods relating to the use of azides in synthesis.

### BACKGROUND

Tetrazoles constitute an important class of heterocyclic compounds enjoying a wide range of applications such as explosives, propellants, organocatalysts, coordination ligands, and “non-classical” isosteres of carboxylic acid functionality in medicinal chemistry. Due to their widespread applications, efforts have gone into developing synthetic methods for tetrazoles, especially the 5-substituted tetrazoles. However, the majority of reported methods find limited use in the large scale synthesis of tetrazoles as they either use explosive or expensive reagents, toxic metals, or excess amounts of azide source. Another concern is the generation of hydrazoic acid (HN₃). HN₃ is very volatile (boiling point: 36° C.) and forms explosive gas phase mixtures with air and/or nitrogen in concentrations as low as 8-15% by volume. HN₃ reacts readily with heavy metals (e.g., Pb, Hg, Cu, and Ag) to form corresponding metal azides that are contact explosives. HN₃ also poses severe health hazards. These safety issues are of great concern for large scale synthesis of tetrazoles, and in general any chemical process involving azides. Thus, improved methods for the synthesis of tetrazoles on a large scale in a safe, efficient, and/or cost effective are needed.

### SUMMARY OF THE INVENTION

Described herein are inventive methods relating to the use of azides in synthesis.

In one aspect, a method is provided. The method comprises reacting in a flow reactor an azide source and a compound of formula (I):

R¹—C≡N (I)

under conditions suitable for forming a compound of formula (II):

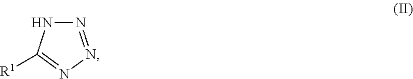

wherein R¹ is alkyl, aryl, heteroalkyl, or heteroaryl, each optionally substituted.

In another aspect, a method is provided. The method comprises reacting an azide source and a compound of formula (I):

R¹—C≡N (I),

under conditions suitable for forming a compound of formula (II):

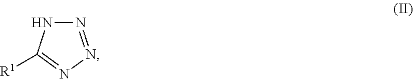

wherein the conditions are selected such that the percent conversion of compound (I) to compound (II) is greater than about 90% in a period of less than 1 hour and in the absence of microwave irradiation, and wherein R¹ is alkyl, aryl, heteroalkyl, heteroaryl, optionally substituted.

In yet another aspect, a method is provided. The method comprises reacting an azide source and a compound of formula (I):

R¹—C≡N (I),

under conditions suitable for forming a compound of formula (II):

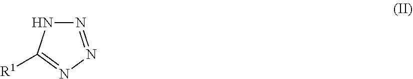

wherein the conditions comprise reacting at a temperature between about 150° C. and about 220° C. and in a solution comprising water and a polar aprotic solvent, wherein the ratio of water to polar aprotic solvent is between 1:9 and 9:1, and wherein R¹ is alkyl, aryl, heteroalkyl, or heteroaryl, each optionally substituted.

Other advantages and novel features of the present invention will become apparent from the following detailed description of various non-limiting embodiments of the invention when considered in conjunction with the accompanying figures. Unless otherwise noted, all references cited herein are incorporated by reference in their entirety. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a comparison of azide reaction in batch and continuous flow processes;

FIG. 2 shows a non-limiting table demonstrating the volumetric throughput of the flow process;

FIGS. 3A and 3B shows photograph of scaled-up synthesis of 5-naphthalen-2-yl-tetrazole in continuous flow using Method B (see Example 2).

### DETAILED DESCRIPTION

Described herein are inventive methods relating the use of azides in synthesis. In some cases, methods are provided for the synthesis of tetrazoles. In some embodiments, the method involves the use of a flow reactor. The methods provided herein are capable of being carried out in short reaction times, with high yields, with minimal side reactions, and/or with minimal chance of explosions due to the presence of azides.

It should be understood that while much of the application herein focuses on the synthesis of tetrazoles, this is by no means limiting, and the methods and systems described herein may be used for other synthetic methods involving azides.

In some embodiments, the present invention provides methods of reacting an azide source and a compound of formula (I):

R¹—C≡N (I);

under conditions suitable for forming a compound of formula (II):

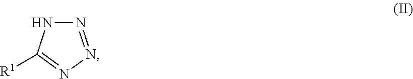

(e.g., a tetrazole), wherein R¹ is alkyl, aryl, heteroalkyl, or heteroaryl, each optionally substituted. In some embodiments, the reaction is carried out under conditions such that the present conversion of compound (I) to compound (II) is greater than about 50%, about 60%, about 70%, about 80%, about 90%, about 92%, about 95%, about 97%, about 98%, about 99%, or greater, in a period of less than about 4 hours, about 3 hours, about 2 hours, about 1 hour, about 50 minutes, about 40 minutes, about 30 minutes, about 20 minutes, about 10 minutes, or less.

In some embodiments, methods provided herein are carried out in the absence of microwave irradiation. It should be understood, that the term “absence of microwave irradiation” does not mean that no microwave irradiation is present while the reaction is being carried out. As will be understood by those or ordinary skill in the art, generally, at least a minimum amount of microwave irradiation (e.g., from outer space) is present in natural circumstances. Accordingly, the absence of microwave irradiation is meant to indicate that no microwave source has been set up and/or directed towards the reaction in such a manner that it would significantly affect the reaction, and/or increase the speed at which the reaction is occurring.

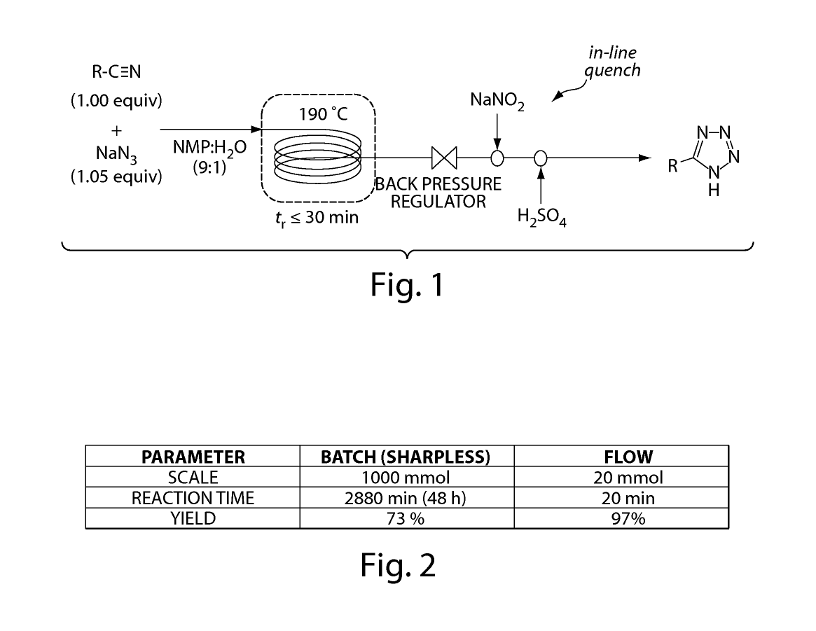

In some embodiments, a method for forming a tetrazole formula (II) is conducted in a flow reactor. Flow reactors will be known to those of ordinary skill in the art. Flow reactors may be provided in various configurations and may be equipped with a number of components to utilize methods described herein. Non-limiting components of a flow reactor include inlet(s) (e.g., for reactants, solvents, quenching agents, etc.), reaction tube and/or chamber (e.g., where the reaction occurs), outlet(s), pressure controller(s) (e.g., back pressure regulators), and temperature control device(s) (e.g., heating device(s) and/or cooling device(s)). A non-limiting example of a flow reactor is shown in FIG. 1 and is described in more detail herein.

In some embodiments, a method of the present invention comprises providing an azide source and a compound of formula (I), to one or more inlets of a flow reactor. The reaction components may be flowed through the flow reactor (e.g., in the reaction tube and/or chamber) for a suitable period of time, during which time, a tetrazole of formula (II) forms. Following the selected amount of time in the flow reactor, the reaction may be quenched with one or more quenching agents (e.g., via an inlet for a quenching reagent), followed by collection of the products and side reactants at one or more outlets of the flow reactor.

Without wishing to be bound by theory, using azides in a flow reactor may prevent and/or reduce the build-up of explosive volatile components which are commonly found in typical syntheses involving azides. For example, due to the lack of head space in the flow reactor, HN₃ may not accumulate in any significant quantity. For example, with batch processes, there is a risk of HN₃ accumulation and condensation in the headspace, a risk of heavy metal azide deposition, a risk of personnel exposure to HN₃, and/or a requirement of a use of a restricted temperature range. In contrast, for continuous flow processes, HN₃ accumulation is minimized in the reactor (e.g., tubular reactor) due to the lack of headspace, a variety of temperature ranges are accessible, including higher ranges as compared to batch processes, and/or the process allows for inline quenching of excess azide source and other hazardous azide waste products.

HN₃ is a known side product which is generally formed when azide reagents are used. HN₃ is a very volatile compound and is reported to form explosive gas phase mixtures with air and/or nitrogen in concentrations as low as 8-15% by volume. Neat HN₃ is extremely explosive, shock sensitive, and highly toxic (e.g., the recommended airborne exposure limit for hydrazoic acid is 0.11 ppm (0.3 mg/m³ as sodium azide) according to the National Institute for Occupational Health and Safety (NIOSH)). Due to the flowing nature of the reaction and the ability to quench any unreacted azide and/or HN₃ prior to removing the reaction mixture from the flow reactor, the risk of potential explosion is greatly reduced and/or eliminated. Thus, the methods of the present invention can minimize and/or eliminate the possibility of build-up of large amounts of HN₃ in either gaseous or liquid form. In some embodiments, the concentration of hydrazoic acid in the reaction flow stream may be less than about 1 M, or less than about 900 mM, or less than about 800 mM, or less than about 700 mM, or less than about 600 mM, or less than about 500 mM, or less than about 400 mM, or less than about 300 mM, or less than about 200 mM, or less than about 100 mM, or less than about 50 mM, or less than about 10 mM, or less than about 5 mM, or less than about 1 mM, or even less.

As mentioned above, in some embodiments of the present invention, a tetrazole of formula (II) may be formed in short reaction periods and/or with good yields. In addition, in some cases, little or no undesired side products are produced in the methods described herein. For example, as shown in Table 1, a typical side product of a tetrazole reaction is the corresponding primary amide. In some cases, the primary amide is formed in less than about 10%, less than about 7%, less than about 5%, less than about 3%, less than about 2%, less than about 1%, less than about 0.05%, less than about 0.03%, less than about 0.01% or less.

In some embodiments, the various conditions of the flow reactor may be selected such that the tetrazole (e.g., a compound of formula (II)) is formed in short reaction times and/or in good yields as described above, and with little production of side reagents. Those of ordinary skill in the art will be able to use the guidelines described herein to select appropriate reaction conditions for the selected reactant without undo experimentations. Non-limiting parameters which may be varied include the solvent selection, the temperature of reaction, the nature of the substituents on the reactant, the presence or absence of a catalyst, and/or the reaction time, variations of which are now described in detail.

In some embodiments of the invention, the reaction is carried out at high temperatures. The use of high temperatures may allow for the reaction to be carried out in shorter periods of time as compared to at lower temperatures. In some embodiments, the reaction may be carried out at a temperature between about 150° C. and about 220° C. In some cases, the reaction may be carried out at a temperature of between about 150° C. and about 210° C., about 160° C. and about 220° C., about 170° C. and about 220° C., about 150° C. and about 200° C., or about 160° C. and about 210° C. In some embodiments, the reaction may be carried out at a temperature of about 150° C., about 160° C., about 170° C., about 180° C., about 190° C., about 200° C., about 210° C., about 220° C., or about 230° C. In some cases, the reaction may be carried out at a temperature below about 230° C., below about 220° C., below about 210° C., below about 200° C., below about 190° C., or below about 180° C.

In some cases, the upper limit of the temperature for the reaction is selected so that the temperature does not reach or exceed the decomposition temperature of the azide source. For example, ZnN₃ becomes unstable above temperatures of about and accordingly, the reaction may carried out at temperatures that do not reach or exceed 240° C. In some cases, the use of a flow reactor can allow for the reaction to be carried out at higher temperatures as compared to non-flow reactor methods without concern for the potential safety issues associated with non-flow reactor methods. For example, in non-flow methods, the temperature may be limited by the build-up of HN₃.

The methods described herein may be carried out in any suitable solvent, including, but are not limited to, non-halogenated hydrocarbon solvents (e.g., pentane, hexane, heptane, cyclohexane), halogenated hydrocarbon solvents (e.g., dichloromethane, chloroform, fluorobenzene, trifluoromethylbenzene), aromatic hydrocarbon solvents (e.g., toluene, benzene, xylene), ester solvents (e.g., ethyl acetate), ether solvents (e.g., tetrahydrofuran, dioxane, diethyl ether, dimethoxyethane.), and alcohol solvents (e.g., ethanol, methanol, propanol, isopropanol, tert-butanol). In certain embodiments, a protic solvent is used. In other embodiments, an aprotic solvent is used. Non-limiting examples of solvents useful include acetone, acetic acid, formic acid, dimethyl sulfoxide, dimethyl formamide, acetonitrile, p-cresol, glycol, petroleum ether, carbon tetrachloride, hexamethyl-phosphoric triamide, triethylamine, picoline, and pyridine. In some embodiments, one or more polar aprotic solvents may be used. Non-limiting examples of solvents include N-methylpyrrolidone (NMP), water, dimethylformamide (DMF), dimethylacetamide (DMA), and dimethylsulfoxide (DMSO). Other solvents will be known to those of ordinary skill in the art. In some embodiments, the reaction is carried out in a solution comprising water and a polar aprotic solvent. In some cases, the ratio of water to a polar aprotic solvent is about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or the like. In some embodiment, the ratio of water to a polar aprotic solvent is between about 9:1 to about 1:9, or between about 10:1 and about 1:10, or between about 8:1 and about 1:8, or between about 7:1 and about 1:7, or between about 10:1 and about 1:1, or between about 9:1 and about 1:1, or between about 15:1 and about 1:1, or between about 12:1 and about 1:1. In a particular embodiment, the ratio of water to a polar aprotic solvent is between about 9:1 and about 1:9. Non-limiting examples of polar aprotic solvents include N-methylpyrrolidone (NMP), dimethylformamide (DMF), dimethylacetamide (DMA), and dimethylsulfoxide (DMSO).

The reaction may be carried out for any suitable period of time. In some cases, the reaction is carried out until the reaction is about 50%, about 60%, about 70%, about 80%, about 90%, about 93%, about 95%, about 96%, about 97%, about 98%, about 99%, about 99.5%, or greater, complete. That is, the reaction is carried out for a period of time until a selected percent of the starting material has been converted into a product. In some cases, the reaction is greater than about 50%, about 60%, about 70%, about 80%, about 90%, about 93%, about 95%, about 96%, about 97%, about 98%, about 99%, about 99.5%, or greater, complete in a period of time of less than about 3 hours, less than about 2 hours, less that about 1 hour, less than about 50 minutes, less than about 40 minutes, less than about 30 minutes, less than about 20 minutes, less than about 15 minutes, less that about 10 minutes, less than about 5 minutes, or less. In a particular, the reaction is greater than about 90% complete in a period of time of less than about 1 hour. In some cases, as described above, the reaction may be carried out such that the amount of side products produced in minimal. In some cases, less than about 20%, less than about 15%, less than about 10%, less than about 8%, less than about 5%, less than about 4%, less than about 3%, less than about 2%, less than about 1%, less than about 0.5%, or less side product is produced.

In some embodiments, the reaction may be carried out in the presence of a catalyst. As will be known to those or ordinary skill in the art, a catalyst may be present to help increase the rate of a reaction and/or to improve the selectivity of a reaction. In some embodiments, the presence of a catalyst may aid in increasing the rate of reaction from the azide to the tetrazole. Non-limiting examples of catalysts include Lewis acids (e.g., ZnBr₂) and tetrabutylammonium fluoride. A catalyst may be provided in an amount of greater than 0.01 equiv, greater than about 0.3 equiv, greater than about 0.5 equiv, greater than about 1 equiv, greater than about 2 equiv, greater than about 3 equiv, greater than about 4 equiv, or even greater. In some embodiments, a catalyst may be provided in an amount of less than about 1 equiv, less than about 0.5 equiv, less than about 0.1 equiv, less than about 0.01 equiv, less than about 0.001 equiv, less than about 0.0001 equiv, or even less. In some embodiments, however, the reaction mixture may be essentially free of a catalyst, wherein the lack of catalyst does not drastically affect the rate of the reaction and/or the selectivity of the reaction. For example, in some cases, a reaction carried out in the absence of a catalyst may occur in essentially the same amount of time and/or with essentially the same selectivity as the reaction carried out in the presence of a catalyst (e.g., ZnBr₂). In some embodiments, the reaction mixture may be essentially free of a Lewis acid catalyst. In some embodiments, the reaction mixture may be essentially free of a Brønsted acid catalyst. For example, in some cases, the reaction may be essentially free of acetic acid.

The azide source may be any suitable source. In some embodiments, the azide source is sodium azide (NaN₃). In some cases, the azide source may be a trialkylammonium azide, a tetraalkylammonium azide, ammonium azide, lithium azide, sodium azide, potassium azide, rubidium azide, cesium azide, beryllium azide, magnesium azide, calcium azide, strontium azide, barium azide, or combinations thereof.

Generally, as will be understood by those of ordinary skill in the art, it is desirable to provide the azide source in as close to one equivalent to the starting material (e.g., a compound of formula (I)) as possible as any access azide source may lead to potentially dangerous side products as well as the azide source itself can be explosive. Accordingly, for the reactions described herein, in some embodiments, a minimal excess of the azide source as compared to the starting material may be provided. In some embodiments, the azide is provided in an amount less than about 4 equiv, less than about 3 equiv, less than about 2 equiv, less than about 1.75 equiv, less than about 1.5 equiv, less than about 1.25 equiv, less than about 1.20 equiv, less than about 1.15 equiv, less than about 1.10 equiv, less than about 1.05 equiv, or less of the starting material (e.g., a compound of formula (I)). In a particular embodiment, the azide source is provided in an amount of about 1.05 equiv. In some embodiments, the azide is provided in an amount of between about 1-4 equiv, between about 1-2 equiv, between about 1-1.75 equiv, between about 1-1.5 equiv, between about 1-1.25 equiv, between about 1-1.1 equiv, or between about 1-1.05 equiv.

A non-limiting example of a flow reactor method of the present invention is as follows. The flow reactor may comprise one or more inlets. In the one or more inlets a compound of formula (I), an azide source, and optionally one or more catalysts, may be provided in a solvent (e.g., a solvent combination comprising water and a polar aprotic solvent, as described herein). The inlets may be connected to the reaction tube and/or chamber. The reaction tube and/or chamber may be associated with one or more temperature control devices such that the temperature in the reaction chamber/tube can be controlled. In some cases, the reaction chamber/tube comprising a metal tubing which is immersed in a heating bath (e.g., oil bath) which is maintained at the desired temperature for the reaction. The reactants may be flowed through the reaction chamber by application or a positive pressure at the inlet (e.g., continued flow of the reactants and/or solvents) and/or a negative pressure at the outlet (e.g., caused by vacuum). While in the reaction chamber/tube, the compound of formula (I) and the azide source may react (e.g., during flow of the solvent through the flow reactor) to form a compound of formula (II). The system may be designed such that the reactants are contained in the flow chamber/tube for the appropriate period of time such that the reaction proceeds to almost completion. The amount of time required will depend on the reaction conditions (e.g., temperature, reactants, solvents, etc.). The residence time in the flow chamber may be controlled using techniques known to those of ordinary skill in the art, including, but not limited to, altering the speed of the flow of the reactant through the flow chamber and/or the length of the reaction tube/chamber, etc. The system may also comprise one or more pressure regulators (e.g., backflow pressure regulator). Once the reaction is complete (or mostly complete) the reaction mixture may be quenched by the addition of a quenching agent. The quenching agent may be provide to the reaction chamber/tube by an inlet. In some cases, the quenching agent may quench any extra azide source and/or any minor amounts of HN₃ produced during the reaction. In some cases, the excess azide source or side products may be quenched by the addition of an acid (e.g., H₂SO₄) or other agent known to quench the azide (e.g., NaNO₂). The reaction mixture may then be collected (e.g., via an outlet) and the product isolated and/or purified, e.g., using techniques known to those of ordinary skill in the art (e.g.,

A variety of tetrazoles (e.g., a compound of formula (II)) may be prepared using the methods described herein. In some embodiments, a chiral tetrazole may be prepared (e.g., see compound 16 in the Examples). For example, in some embodiments, a tetrazole may be prepared having an enantiomeric excess greater than about 50%, greater than about 60%, greater than about 70%, greater than about 80%, greater than about 90%, greater than about 95%, greater than about 98%, or greater than about 99%. In some embodiments, the tetrazole may be a racemic mixture.

The R¹ group on the compound of formula (I) may be any suitable group. For example, R¹ may be an alkyl group, an aryl group, a heteroalkyl group, or a heteroaryl group, all optionally substituted. In some embodiments, the R¹ group may be a substituted or unsubstituted, branched or unbranched, cyclic or acyclic C₁₋₃₀ aliphatic; a substituted or unsubstituted, branched or unbranched, cyclic or acyclic C₁₋₃₀ heteroaliphatic; substituted or unsubstituted aryl; or a substituted or unsubstituted heteroaryl group. Non-limiting examples of suitable R¹ groups include 4-methoxyphenyl; phenyl; naphthalene-2-yl; 4-benzaldehyde; 4-nitrophenyl; m-tolyl; 2-pyrazinyl; 2-pyridinyl; 6-methoxyquinoline; biphenyl-3-ol; p-tolyl; o-tolyl; 4-phenol; 3′-biphenyl-4-ol; 4′-biphenyl-4-ol; and (S)-2-pyrrolidine-1-carboxylic acid benzyl ester.

### Definitions

For convenience, certain terms employed in the specification, examples, and appended claims are listed here.

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in “Organic Chemistry,” Thomas Sorrell, University Science Books, Sausalito: 1999, the entire contents of which are incorporated herein by reference. As used herein, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The alkyl groups may be optionally substituted, as described more fully below. In some cases, the alkyl group may be a lower alkyl group, i.e., an alkyl group having 1 to 10 carbon atoms (e.g., methyl, ethyl, propyl, butyl, pentyl, hexyl, heptyl, octyl, nonyl, or decyl). In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C₁-C₁₂ for straight chain, C₃-C for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, butyl, isobutyl, tert-butyl, 2-ethylhexyl, cyclopropyl, cyclobutyl, cyclopentyl, cyclohexyl, and the like, each optionally substituted.

The terms “alkenyl” and “alkynyl” refer to unsaturated aliphatic groups analogous to the alkyl groups described above, but containing at least one double or triple bond respectively. Alkenyl and alkynyl groups may be analogous in length and possible substitutions to the alkyls described above, but contain at least one double or triple bond, respectively. The “heteroalkenyl” and “heteroalkynyl” refer to alkenyl and alkynyl groups as described herein in which one or more atoms is a heteroatom (e.g., oxygen, nitrogen, sulfur, and the like).

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, allyl, n-butyl, sec-butyl, isobutyl, t-butyl, n-pentyl, sec-pentyl, isopentyl, t-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “cycloalkyl,” as used herein, refers specifically to groups having three to ten, preferably three to seven carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or hetercyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

“Heteroalkyl” groups are alkyl groups wherein at least one atom is a heteroatom (e.g., oxygen, sulfur, nitrogen, phosphorus, etc.), with the remainder of the atoms being carbon atoms. Examples of heteroalkyl groups include, but are not limited to, alkoxy, poly(ethylene glycol)-, alkyl-substituted amino, tetrahydrofuranyl, piperidinyl, morpholinyl, etc., each optionally substituted.

The term “aryl” is given its ordinary meaning in the art and refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls and/or heterocyclyls. The aryl group may be optionally substituted, as described herein. Substituents include, but are not limited to, any of the previously mentioned substitutents, i.e., the substituents recited for aliphatic moieties, or for other moieties as disclosed herein, resulting in the formation of a stable compound. In some cases, an aryl group is a stable mono- or polycyclic unsaturated moiety having preferably 3-14 carbon atoms, each of which may be substituted or unsubstituted. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups.

The terms “heteroaryl” is given its ordinary meaning in the art and refers to aryl groups comprising at least one heteroatom as a ring atom. A “heteroaryl” is a stable heterocyclic or polyheterocyclic unsaturated moiety having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In some cases, a heteroaryl is a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like.

It will also be appreciated that aryl and heteroaryl moieties, as defined herein may be attached via an alkyl or heteroalkyl moiety and thus also include -(alkyl)aryl, -(heteroalkyl)aryl, -(heteroalkyl)heteroaryl, and -(heteroalkyl)heteroaryl moieties. Thus, as used herein, the phrases “aryl or heteroaryl moieties” and “aryl, heteroaryl, -(alkyl)aryl, -(heteroalkyl)aryl, -(heteroalkyl)heteroaryl, and -(heteroalkyl)heteroaryl” are interchangeable. Substituents include, but are not limited to, any of the previously mentioned substituents, i.e., the substituents recited for aliphatic moieties, or for other moieties as disclosed herein, resulting in the formation of a stable compound.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂F; —CHF₂; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments described herein.

It will be appreciated that the above groups and/or compounds, as described herein, may be optionally substituted with any number of substituents or functional moieties. That is, any of the above groups may be optionally substituted. As used herein, the term “substituted” is contemplated to include all permissible substituents of organic compounds, “permissible” being in the context of the chemical rules of valence known to those of ordinary skill in the art. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted heteroalkyl” must still comprise the heteroalkyl moiety and can not be modified by substitution, in this definition, to become, e.g., an alkyl group. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

Examples of substituents include, but are not limited to, alkyl, aryl, aralkyl, cyclic alkyl, heterocycloalkyl, hydroxy, alkoxy, aryloxy, perhaloalkoxy, aralkoxy, heteroaryl, heteroaryloxy, heteroarylalkyl, heteroaralkoxy, azido, amino, halogen, alkylthio, oxo, acylalkyl, carboxy esters, carboxyl, -carboxamido, nitro, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, alkoxyalkyl, perhaloalkyl, arylalkyloxyalkyl, and the like.

Also used herein, “azide” is given its ordinary meaning in the art and refers to a functional group having the formula —N₃.

Also as used herein, “tetrazole” is given its ordinary meaning in the art and refers to a heterocyclic functional group having a five-membered ring structure where four of the atoms that form the ring are nitrogen and one of the atoms that forms the ring is carbon.

These and other aspects of the present invention will be further appreciated upon consideration of the following Examples, which are intended to illustrate certain particular embodiments of the invention but are not intended to limit its scope, as defined by the claims.

### EXAMPLE 1

With a goal to identify reaction conditions suitable for a continuous flow process, several reported conditions for tetrazole synthesis were screened in small-scale batch microwave reactions, using 4-methoxy-benzonitrile (1) as a model substrate (Table 2). Generally, reactions optimized in small-scale batch microwave processes can be easily reproduced in the continuous-flow microreactors. The conditions initially reported by Sharpless and coworkers, and later shown to be effective under microwave irradiation by Fang and coworkers (Table 2, entries 4-6) showed promise for the flow process development, albeit with some modifications. The highest yield (65%) was obtained at 140° C. and 30 min hold time under microwave, with the reaction remaining homogenous throughout its entire course. Although the yield improved at higher temperatures, but an undesired byproduct, carboxamide (1b) resulting from nitrile hydration was also obtained (9% yield).

TABLE 1

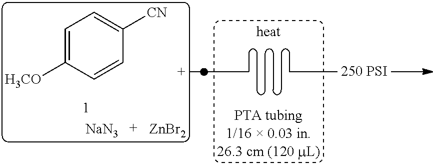

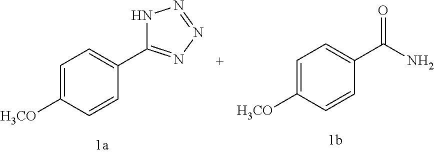 [SMILES: COc1ccc(-c2nnn[nH]2)cc1 ; COc1ccc(C(N)=O)cc1]

| | | | | | % |
|---|---|---|---|---|---|
| | | NaN₃ | ZnBr₂ | T | Yield^{(a)} |
| Entry | Solvent | (equiv.) | (equiv.) | (° C.) | 1a (1b) |
| 1 | THF:H₂O (1:4) | 4 | 2 | 170 | 76 (10) |
| 2 | IPA:H₂O (1:4) | 4 | 2 | 190 | 64 (11) |
| 3 | NMP:H₂O (1:4) | 4 | 2 | 190 | 83 (8) |
| 4 | NMP:H₂O (1:4) | 2 | 1 | 190 | 72 (7) |
| 5 | NMP:H₂O (1:4) | 1.05 | 0.5 | 190 | 56 (4) |
| 6 | NMP:H₂O (9:1) | 1.05 | 0.5 | 190 | 61 (none) |
| 7 | NMP:H₂O (9:1) | 1.05 | 0.5 | 200 | 63 (trace) |

^{(a)}Determined by reverse phase HPLC method using naphthalene as an external standard. All the reactions were carried out in 0.2M concentration with respect to 1 (residence time t_{R} = 20 min).

TABLE 2

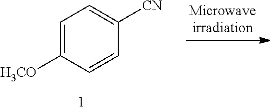

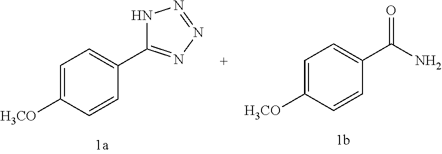 [SMILES: COc1ccc(-c2nnn[nH]2)cc1 ; COc1ccc(C(N)=O)cc1]

| | Reagents | | T | Time | % Yield^{(a)} |
|---|---|---|---|---|---|
| Entry | (equiv.) | Solvent | ° C. | min | 1a (1b) |
| 1 | NaN₃ (12), | DMF | 120 | 15 | 93 |
| | NH₄Cl (12) | | | | |
| 2 | NaN₃ (1.2), | DMF | 120 | 15 | trace |
| | NH₄Cl (1.2) | | | | |
| 3 | TMSN₃ (1.2), | THF | 100 | 30 | — |
| | TBAF (0.5) | | | | |
| 4 | NaN₃ (4), | THF:H₂O ^{(b)} | 100 | 30 | 10 |
| | ZnBr₂ (2) | | | | |
| 5 | NaN₃ (4), | THF:H₂O ^{(b)} | 120 | 30 | 45 (3) |
| | ZnBr₂ (2) | | | | |
| 6 | NaN₃ (4), | THF:H₂O ^{(b)} | 140 | 30 | 65 (9) |
| | ZnBr₂ (2) | | | | |

^{(a)}Yields obtained by reverse phase HPLC method using naphthalene as a standard. All the reactions were carried out in 0.1M concentration with respect to 1.
^{(b)} THF:H₂O (1:4).

To test the conditions in entry 6 (Table 2) in a continuous flow setup, a solution of reactants (1, NaN₃ and ZnBr₂) was pumped into a PFA microtube ( 1/16×0.03 in; 120 μL capacity) immersed in an oil bath (heating source), and equipped with a 250 psi back pressure regulator at its distal end. The reaction when carried out at 170° C. with a residence time (t_{R}) of 20 min, gave the product in 76% yield (Table 1, entry 1). The yields were found to decrease when the co-solvent THF was replaced with isopropyl alcohol (IPA) in order to access higher temperatures (Table 1, entry 2). Use of N-methylpyrrolidone (NMP) as a co-solvent along with water, however, gave the product in 83% yield at 190° C. Reducing the equivalents of NaN₃ and ZnBr₂ in a stepwise manner decreased the product yield (Table 1, entries 4 & 5). However, changing the solvent polarity from largely aqueous (1:9; NMP:H₂O) to largely non-aqueous (9:1 NMP:H₂O) improved the yield (61%), and importantly, no hydrolysis product (1b) was observed under these conditions (Table 1, entry 6). Increasing the reaction temperature to 200° C. and t_{R} to 30 min did not improve the yield significantly but instead, lead to the reappearance of the undesired byproduct (Table 1, entry 7).

In order to expand the scope of this flow method, a variety of nitriles were subjected to the optimized conditions in the entry 6 (Table 1), and the results are summarized in Tables 3 and 4 (Method A). For substrates 2 and 3, where no electron-donating or electron-withdrawing group was present on the aromatic ring, or substrate 4 where the nitrile was rendered electron poor by the presence of electron-withdrawing group at the para position, the reactions proceeded to 100% conversions without the formation of any side product. Similarly, meta tolunitrile (5) and the heteroaromatic substrates (6-8) also showed excellent conversions. Electron rich nitriles 10 and 10 reacted to give moderate but clean conversions to the corresponding tetrazoles. The biphenyl nitriles 9, 13 and 14 also proved to be good substrates for this reaction regardless of position of the phenolic hydroxy group on the second aromatic ring. Notably, chiral nitrile 15 provided 15a, a derivative of which (no CBZ group) has found utility as an organocatalyst, in >99% ee and 92% yield based on conversion. To test if an increase in the t_{R} can drive the reaction of moderately yielding substrates to completion, the model substrate 1 was reacted at a t_{R} of 30 min (Table 4). There was no significant change in the conversion observed; instead a small amount of hydrolysis product 1a was formed. However, significant improvement in the reaction rate was observed by doubling the concentration of the reaction (0.4 M). For nitrile 1, the conversion increased from 65% to 81% (t_{R}=30 min), while similar improvement in conversions were observed for substrates 11 and 13-15 when the reaction concentration was doubled.

As this continuous flow method has the advantage of using high temperatures in a safe manner, it was determined that the presence of a catalyst (e.g., ZnBr₂) was not essential for all reactions carried out at these temperatures. To test this, the flow process was repeated with selected substrates without the use of ZnBr₂ (Tables 3 and 4, Method B). The non-substituted benzo- and napthonitrile substrates (2 and 3), electron poor nitrile (4), and the heterocyclic substrates (6, 7 and 8), all showed excellent conversions to corresponding tetrazoles in the absence of ZnBr₂. The conversions were found to decrease moderately in case of biphenyl substrates 9, 13 and 14 indicating decrease in the reaction rate of these substrates in the absence of ZnBr₂. Similar decrease in conversion was observed for the electron rich substrates (1 and 12), but it was noted that there was no side product observed in the absence of ZnBr₂ even at 30 min of residence time. This shows that ZnBr₂ may be promoting the competing side reaction. Thus, the use of ZnBr₂ may be useful for enhancing the conversion of the electron-rich nitriles, but can also lead to formation of side product. In many, if not all instances, reactions without ZnBr₂ can give clean conversions.

TABLE 3

| | | Method A | | Method B | |
|---|---|---|---|---|---|
| | | (with ZnBr₂) | | (w/o ZnBr₂) | |
| | | conversion | | conversion | |
| tetrazole product | | (%) ^{a} | yield (%) ^{a} | (%) ^{a} | yield (%) ^{a} |
| 2a | 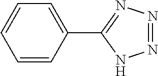 [SMILES: c1ccc(-c2nnn[nH]2)cc1] | 100 | 98 | 100 | 98 (96) ^{b} |
| 3a | 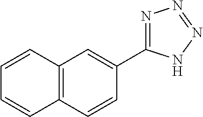 [SMILES: c1ccc2cc(-c3nnn[nH]3)ccc2c1] | 100 | 96 | 99 | 95 (94) ^{b} |
| 4a | 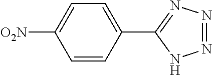 [SMILES: O=[N+]([O-])c1ccc(-c2nnn[nH]2)cc1] | 100 | 96 (93) ^{b} | 98 | 95 |
| 5a | 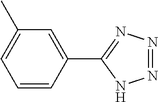 | 93 | 87 | 90 | 85 (81) ^{b} |
| 6a | 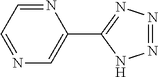 [SMILES: c1cnc(-c2nnn[nH]2)cn1] | 100 | 97 | 94 | 93 (90) ^{b} |
| 7a | 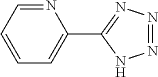 [SMILES: c1ccc(-c2nnn[nH]2)nc1] | 100 | 94 | 100 | 96 (94) ^{b} |
| 8a | 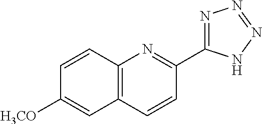 [SMILES: COc1ccc2nc(-c3nnn[nH]3)ccc2c1] | 100 | 96 | 99 | 98 (97) ^{b} |
| 9a | 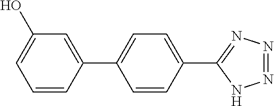 [SMILES: Oc1cccc(-c2ccc(-c3nnn[nH]3)cc2)c1] | 97 | 93 (91) ^{b} | 81 | 79 |

[nitrile]₀ = 0.2M;
reaction temperature = 190° C.;
tᵣ = 20 min.
^{a} Determined by HPLC.
^{b} Isolated yield.

TABLE 4

| | | Method A (with ZnBr₂) | | | Method B (w/o ZnBr₂) | |
|---|---|---|---|---|---|---|
| | | conv. | yield a | yield b | conv. | yield a |
| tetrazole product | | (%) ^{a} | (%) ^{a} | (%) ^{a} | (%) ^{a} | (%) ^{a} |
| 1a | 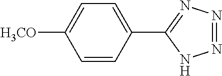 [SMILES: COc1ccc(-c2nnn[nH]2)cc1] | 63 ^{c} 86 ^{d} | 61 78 (73) ^{b} | <1 7 ^{c} | — 83 ^{d} | — 81 (79) ^{b} |
| 10a | 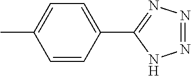 | 64 | 61 | 0 | 61 | 57 (51) ^{b} |
| 11a | 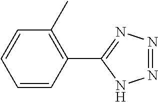 | 32 | 28 | 0 | 25 | 25 (23) ^{b} |
| 12a | 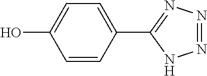 [SMILES: Oc1ccc(-c2nnn[nH]2)cc1] | 50 56 ^{d} | 48 53 | 0 1 | — 49 ^{d} | — 48 (45) ^{b} |
| 13a | 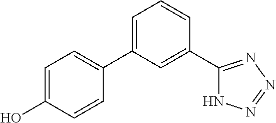 [SMILES: Oc1ccc(-c2cccc(-c3nnn[nH]3)c2)cc1] | 97 | 93 (91) ^{b} | 0 | 86 | 84 |
| 14a | 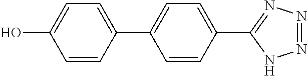 [SMILES: Oc1ccc(-c2ccc(-c3nnn[nH]3)cc2)cc1] | 91 | 86 (86) ^{b} | 0 | 82 | 77 |
| 15a | 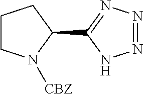 | 82 | 75 (70) ^{b, e} | 0 | 71 ^{e} | 65 |

Unless otherwise noted [nitrile]₀ = 0.4M;
reaction temperature = 190° C;
tᵣ = 20 min.
^{a} Determined by HPLC.
^{b} Isolated yield.
^{c} [nitrile]₀ = 0.2M,
^{d} tᵣ = 30 min.
^{d} ee > 99% (HPLC).

To demonstrate the scale-up capabilities of this, the synthesis of 3a was carried out using aUniqsis FlowSyn continuous flow reactor. FlowSyn is an integrated continuous flow reactor system that uses a pair of high pressure pumps to deliver reagent solutions through a ‘T’-mixer into the electrically heated flow coil or column reactors. The homogenous solution of reagents ([3]=1M; [NaN₃]=1.05 M) in NMP:H₂O (7:3) was pumped using a single pump through a coiled PFA tubing reactor (volume of heated zone˜6.9 mL) with a flow rate of 0.35 mL/min (tᵣ=20 min) at 190° C. (see Example 2). The flow process was run continuously for 2.5 h to obtain 9.7 g of 3a in 96% yield. This corresponds to a product output of 4.85 g/h or 116 g/day for the tetrazole 3a.

Overall, the method performed is a safer alternative for currently used methods to synthesize 5-substituted tetrazoles as the hazards due to accumulation and condensation of HN₃ are greatly minimized. Only uses a slight excess of NaN₃ (1.05 equiv) was used, and hence the production of azide waste is minimal. The method is highly efficient and clean, and works for a wide range of substrates. In case of substrates where the reaction does not go to completion, the remaining NaN₃ in the reaction can be quenched by introducing streams of sodium nitrite and sulfuric acid after the reaction is complete. The incorporation of this quenching procedure increases the overall safety of the process. Therefore, given the widespread applications of tetrazoles in chemical and pharmaceutical industry, this method can serve as a safe and highly efficient alternative for synthesis of tetrazoles.

### EXAMPLE 2

This example provides additional experimental details and data in connection with Example 1.

### Materials and General Methods:

Commercially available reagents were obtained from Aldrich Chemical Co. (St. Louis, Mo.) and used without any further purification. Reagent grade solvents were used for all non-aqueous extractions. Reactions were monitored by analytical thin-layer chromatography using EM silica gel 60 F-254 pre-coated glass plates (0.25 mm). ¹H and ¹³C NMR spectra were recorded at 25° C. on a Bruker-Avance 400 (400 MHz) spectrometer and compared to known literature compounds if available. HPLC analysis was performed on an Agilent 1200 Series LC/MS using an Eclipse XDB-C18 reverse phase column, and gradient elution with acetonitrile/water mobile phase. The detector signals at 254 or 210 nm were monitored. Yields were calculated by external standard method using naphthalene as a standard. Batch microwave reactions were carried out using Biotage Initiator single cavity microwave reactor using 0.5-2 mL sealed vials under high absorption range.

### General Protocol for Batch Microwave Reactions:

Nitrile 1 (0.1 mmol), the azide source and other reagents were combined in a 0.5-2 mL (5 mL total volume) microwave vial and diluted to 1 mL with the solvent (Table 1). The vial was then sealed, placed in the microwave cavity, and irradiated at high absorption for the specified time and temperature. The reaction vial was allowed to cool to the room temperature, the reaction mixture was diluted in saturated NaHCO₃ (30 mL) and washed with ethyl acetate. The water phase was acidified to pH<1 with concentrated HCl and extracted with ethyl acetate. The combined organic layers were dried, and concentrated in vacuo to give the product.

### General Protocol for Continuous Flow Synthesis of Tetrazoles (Method A):

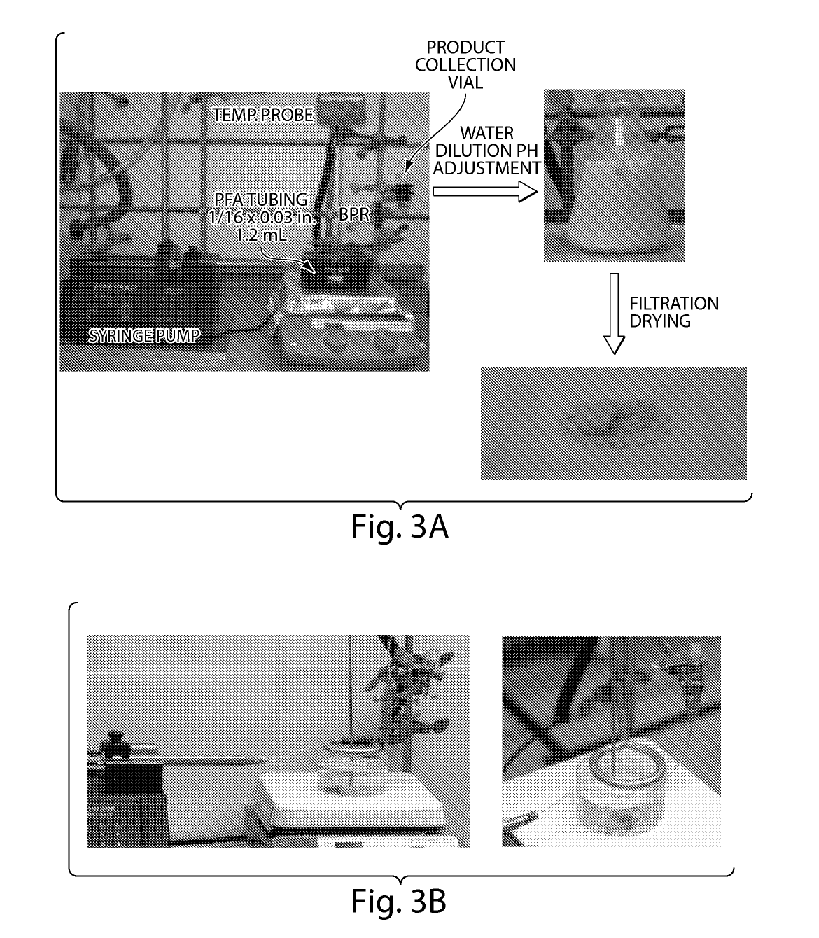

Sodium azide (68 mg, 1.05 mmol) was added to a solution of zinc bromide (111 mg, 0.5 mmol) in 0.5 mL water. To this solution was added the nitrile substrate (1 mmol) dissolved in 4.5 mL of N-methylpyrrolidone (NMP) and the resulting clear solution was filled in a 10 mL stainless steel syringe (Harvard Apparatus, High Pressure stainless steel syringe with 1/16 inch SWAGELOK®), which was then charged to a syringe pump (Harvard PHD 2000) (see FIGS. 3A and 3B). The syringe was connected to a 41 cm tubular coiled reactor (Upchurch Scientific PFA tubing; 1/16×0.03 in.) with the help of SWAGELOK® fittings (stainless steel front ferrule, stainless steel back ferrule and 316 stainless steel nut for 1/16 in.). The tubular reactor was coiled in such a way that 26.4 cm (constituting an internal volume of 120 μL) of the middle portion of the tubing was dipped in the oil bath (heating source). A 250 psi back pressure regulator (Upchurch Scientific) was installed at the distal end of this tubing followed by short outlet tubing. The reaction mixture was pumped through the tubular reactor at a rate of 6 μL/min, and the temperature of the oil bath was set to 190° C. This resulted in a 20 min residence time for reaction mixture in the part of tubing immersed in the oil bath. After the reactor reached equilibrium (flowing approximately 3 full-reactor volumes), 1 mL of output was collected and diluted with 10 mL of water. The solution was acidified to pH 1 using 3 N HCl and stirred vigorously. Note: for the heterocyclic substrates 10, 11 and 12, the reaction was basified and stirred vigorously until a white precipitate (zinc hydroxide) was observed. The precipitate was filtered and the pH of the filtrate was adjusted to 6.5. The resulting precipitate was extracted with ethyl acetate, and the organic layer concentrated to yield the product. A white precipitate appeared which was extracted into non-aqueous layer using 10 mL of ethyl acetate. The organic layer was separated and concentrated to yield a crude product. This crude product was taken in 20 mL of 0.25 N sodium hydroxide solution and stirred vigorously until a white precipitate of zinc hydroxide was observed. The resulting precipitate was filtered and the filtrate was acidified to pH 1 using 3 N HCl. The tetrazole product precipitated upon stirring, which was then filtered, washed with 10 mL of 3 N HCl, and dried to obtain a pure product.

The parts used to assemble the apparatus shown in FIG. 3B include at least the following: PHD 22/2000 Remote Syringe Pump from Harvard Apparatus (syringe rack high pressure); PFA tubing ( 1/16×0.03 inch) from Upchurch Scientific; SWAGELOK® fitting from Upchurch® Scientific (Stainless Steel Nut for 1/16 inch; Stainless Steel Front Ferrule; Stainless Steel Back Ferrule); 250 psi Back Pressure Regulator from Upchurch® Scientific (or alternatively, a 100 psi PEEK™ back pressure regulator); Flat Bottom Fitting from Upchurch® Scientific; Nut (PEEK™); Flangeless Ferrule (TEFZEL®); High Pressure 8 mL Stainless Steel Syringe with 1/16 inch SWAGELOK®; 5/16 Wrenches (2); Aluminum or any other flexible wire; Glass Vials; Oil Bath with High Temperature Oil and a Magnetic Stirrer-1

### General Protocol for Continuous Flow Synthesis of Tetrazoles (Method B):

Sodium azide (138 mg, 2.1 mmol) was dissolved in 0.5 mL of water and added to the nitrile substrate (2 mmol) dissolved in 4.5 mL of NMP. The resulting solution was filled in a 10 mL stainless steel syringe and the flow process was carried out as described for Method A. The flow process was carried out behind an explosion shield for personal safety. To determine the isolated yield, 4 mL of the post-reaction stream was diluted with 100 mL of water. The solution was acidified to pH 1 (except for the heterocyclic substrates 5, 6 and 7 (Table 2) using 3 N HCl solution and stirred vigorously for 30 min. A white precipitate appeared which was extracted into non-aqueous layer using 50 mL of ethyl acetate. The organic layer was separated and concentrated to yield a pure product. For heterocyclic substrates 5, 6 and 7, the reaction was diluted with water and the pH was adjusted to 6.5 with vigorous stirring. The resulting precipitate was filtered, washed with 15 mL of 3N HCl solution and dried to yield pure product.

Procedure for Scaled-Up Synthesis of Tetrazole in Continuous Flow using Method B:

Napthelene-2-carbonitrile (3) (3.06 gm, 20 mmol) was dissolved in 45 mL of NMP. To this solution was added sodium azide (1.36 gm, 21 mmol) in 5 mL water resulting in a clear homogenous solution. A part of this solution (8 mL) was filled in a 10 mL stainless steel syringe (Harvard Apparatus, High Pressure stainless steel syringe with 1/16 inch SWAGELOK®) and charged to a syringe pump (Harvard PHD 2000). The syringe was connected to a 290 cm tubular coiled reactor (Upchurch Scientific PFA tubing; 1/16×0.03 in.) with the help of SWAGELOK® fittings (stainless steel front ferrule, stainless steel back ferrule and 316 stainless steel nut for 1/16 in.). The tubular reactor was coiled in such a way that 264 cm (constituting an internal volume of 1.20 mL) of the middle portion of the tubing was dipped in the oil bath (heating source). A 200 psi back pressure regulator (Upchurch Scientific) was installed at the distal end of this tubing followed by short outlet tubing. The reaction mixture was pumped through the tubular reactor at the rate of 60 μL/min, and the temperature of the oil bath was set to 190° C. This resulted in a 20 min residence time for reaction mixture in the part of tubing immersed in the oil bath. After the reactor reached equilibrium (flowing approximately 3 full-reactor volumes), the post-reaction stream was collected for 12 hours (Due to instrument limitations, the syringe had to be refilled after every 2 hours with the reaction mixture. The empty syringe was displaced with a syringe charged with the reaction mixture by halting the flow for approximately one minute. However, this did not affect the efficiency of the process.). The collected volume (43.2 mL) was the acidified to pH 1 using 3 N HCl and stirred for 30 min to obtain a light yellow precipitate (FIG. 3). The precipitate was filtered and dried overnight to obtain 3.28 g of pure product (isolated yield=97%).

Large Scale Synthesis of 5-Naphthalen-2-yl-tetrazole using Uniqsis FlowSyn™ Continuous Flow Reactor:

FlowSyn™ is an integrated continuous flow reactor system that uses a pair of high pressure piston pumps to deliver reagent solutions through a ‘T’-mixer into the electrically heated flow coil or column reactors.

For this process, the reagent solution was pumped through a single pump into a coiled reactor consisting of PFA tubing ( 1/16×0.03 inch ID) accommodating a volume of 6.9 mL (heated portion of the tubular reactor). A 100 psi back pressure regulator was installed at the distal end of the tubing. The reagent solution was prepared by dissolving 11.4 g (7.5 mmol) of napthelene-2-carbonitrile 3 in 56 mL of NMP. To this solution was added the solution of NaN₃ (5.1 g) dissolved in 19 mL of water. It should be noted that solvent ratio of the solvent NMP:Water was changed from 9:1 to approximately 7.5:2.5 in order to account for the solubility of NaN₃ in water. The solution of reactants was then pumped through the PFA coil reactor at a rate of 0.345 μL/min (residence time tᵣ=20 min) at 190° C. The product collection was started after flowing two full reactor volumes in order to achieve the steady state equilibrium. The flow process was run continuously for 2.5 hours. The reaction mixture was then diluted with 500 mL of water and acidified to pH 1 using 3 N HCl with stirring. The resulting precipitate was filtered, washed with 1 N HCl, and dried in air to obtain 9.7 g of pure product in 96% isolated yield. This flow process gives the product output of 4.85 g/h or 116 g/day for the tetrazole 3a (see Table 5).

TABLE 5

| | Reaction | Reactor | | | Isolated | Product |
|---|---|---|---|---|---|---|
| Product | conc. | Volume | Temp. | Flow Rate | Yield | Output |
| 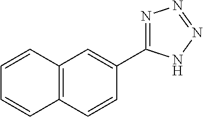 [SMILES: c1ccc2cc(-c3nnn[nH]3)ccc2c1] | 1M | 6.9 mL | 190° C. | 0.35 mL/min (tᵣ~20 min) | 9.7 g in 2.5 h (96%) | 4.85 g/h or 116 g/day |

### Ferric Chloride Test for Detection of Hydrazoic Acid in the Post-Reaction Stream:

Strips of filter paper were soaked in a 10 uM (micromolar), 1 mM and 100 mM solution of iron (III) chloride and dried. After being placed on the top of the in-process post reaction stream collection for 2 hours, no change in the color was observed. As a positive control, 3M HCl was added drop-wise to a vial containing 1M NaOH solution in presence of the strips. The strips showed color change to bright red within a minute. The test was repeated on the post-reaction stream of a reaction containing no substrate at 1M concentration of NaN₃ with the test showing negative results. The results thus indicates that the post reaction streams are free of HN₃ contamination.

### Spectral Data:

5-(4-Methoxy-phenyl)-tetrazole (1a): ¹H NMR (400 MHz, DMSO-d₆) δ 7.98 (d, J=9.2 Hz, 2H) 7.16 (d, J=8.8 Hz, 2H), 3.84 (s, 3H); ¹³C NMR (100 MHz, DMSO-d₆) δ 161.4, 154.3, 128.5, 114.8, 55.4; HRMS-ESI m/z calculated for C₈H₉N₄O [M+H]⁺: 177.0771, found: 177.0763

5-Phenyltetrazole (2a): ¹H NMR (400 MHz, DMSO-d₆) δ 8.04-8.03 (m, 2H), 7.61-7.59 (m, 3H); ¹³C NMR (100 MHz, DMSO-d₆) δ 155.2, 131.2, 129.3, 126.9, 124.0; HRMS-ESI m/z calculated for C₇H₇N₄ [M+H]⁺ 147.0665, found 147.0664

5-Naphthalen-2-yl-tetrazole (3a): ¹H NMR (400 MHz, DMSO-d₆): δ 8.66 (m, 1H), 8.15-8.08 (m, 3H), 8.03-8.01 (m, 1H), 7.65-7.61 (m, 2H); ¹³C NMR (100 MHz, DMSO-d₆): δ 155.6, 133.8, 132.5, 129.1, 128.5, 127.8, 127.5, 127.1, 126.9, 123.6, 121.6; HRMS-ESI m/z calculated for C₁₁H₈N₄ [M−H]⁻ 195.0676, found 195.0676

5-(4-Nitrophenyl)tetrazole (4a): ¹H NMR (400 MHz, DMSO-d₆): δ 8.44 (d, J=8.4 Hz, 2H); 8.30 (d, J=8.4 Hz, 2H); ¹³C NMR (100 MHz, DMSO-d₆) δ 155.2, 148.5, 130.7, 132.5, 128.0, 124.5; HRMS-ESI m/z calculated for C₇H₅N₅O₂ [M−H]⁻ 190.0370, found 190.0368

5-m-Tolyl-tetrazole (5a): ¹H NMR (400 MHz, DMSO-d₆): δ 7.86 (s, 1H); 7.82 (d, J=8.0 Hz, 1H), 7.46 (t, J=8.0 Hz, 1H), 7.37 (d, J=7.8 Hz,1H), 2.40 (s, 3H); ¹³C NMR (100 MHz, DMSO-d₆) δ 154.4, 138.5, 129.1, 127.2, 123.2, 20.4; HRMS-ESI m/z calculated for C₈H₈N₄ [M+H]⁺ 161.0822, found 161.0816

2-(Tetrazol-5-yl)-pyrazine (6a): ¹H NMR (400 MHz, DMSO-d₆): δ 9.41 (d, J=0.8 Hz, 1H); 8.89 (m, 2H); ¹³C NMR (100 MHz, DMSO-d₆) δ 155.1, 146.8, 144.8, 143.4, 140.1; HRMS-ESI m/z calculated for C₅H₄N₆ [M−H]⁻ 147.0425, found 147.0423

2-(Tetrazol-5-yl)-pyridine (7a): ¹H NMR (400 MHz, DMSO-d₆): δ 8.79 (d, J=4.4 Hz, 1H), 8.22 (d, J=8.0 Hz, 1H), 8.10-8.05 (m, 1H), 7.64-7.61 (m, 1H); ¹³C NMR (100 MHz, DMSO-d₆): δ 154.9, 150.0, 143.9, 138.1, 125.9, 122.5; HRMS-ESI m/z calculated for C₆H₅N₅ [M−H]⁻ 146.0472, found 146.0472

6-Methoxy-2-(tetrazol-5-yl)-quinoline (8a): ¹H NMR (400 MHz, CD₃OD): δ 8.42 (d, J=8.0 Hz, 1H), 8.24 (d, J=8.0 Hz, 1H), 8.06 (d, J=8.0 Hz, 1H), 7.48 (dd, J=9.2 Hz, J=2.8 Hz, 1H), 7.37 (d, J=2.8 Hz, 1H), 3.98 (s, 3H). ¹³C NMR (100 MHz, CD₃OD): δ 159.5, 154.6, 144.0, 136.7, 130.6, 130.5, 123.7, 119.4, 105.1, 55.0. HRMS-ESI m/z calcd for C₁₁H₉N₅O [M+H]⁺: 228.0885, found: 228.0887

4′-(Tetrazol-5-yl)-biphenyl-3-ol (9a): ¹H NMR (400 MHz, CD₃OD): δ 8.02 (d, J=8.0 Hz, 2H); 7.75 (d, J=8.4 Hz, 2H); 7.23 (t, J=8.0 Hz, 1H); 7.10-7.04 (m, 2H); 6.78-6.75 (m, 1H). ¹³C NMR (100 MHz, CD₃OD): δ 159.2, 157.3, 145.7, 142.4, 133.7, 131.1, 129.0, 128.9, 128.7, 119.3, 116.2, 114.9. HRMS-ESI m/z calculated for C₁₃H₁₁N₄O [M+H]⁺: 239.0927, found 239.0933.

5-(p-Tolyl)-tetrazole (10a): ¹H NMR (400 MHz, DMSO-d₆): δ 7.93 (d, J=8.4 Hz, 2H) 7.41 (d, J=8.4 Hz, 2H), 2.39 (s, 3H); ¹³C NMR (100 MHz, DMSO-d₆): δ 154.9, 141.1, 129.9, 121.2, 21.0; HRMS-ESI m/z calculated for C₈H₈N₄ [M+H]⁺: 161.0822; found: 161.0827.

5-o-Tolyl-tetrazole (11a): ¹H NMR (400 MHz, CD₃OD): δ 7.55-7.53 (m, 1H), 7.34-7.31 (m, 2H), 7.27-7.23 (m, 1H), 2.45 (s, 3H); ¹³C NMR (100 MHz, DMSO-d₆): δ 137.2, 130.2, 128.3, 127.8, 125.0 HRMS-ESI m/z calculated for C₈H₉N₄ [M+H]⁺ 161.0822, found 161.0828

4-(Tetrazol-5-yl)-phenol (12a): ¹H NMR (400 MHz, DMSO-d₆): δ 10.1 (broad s, 1H), 7.85 (d, J=8.8 Hz, 2H); 6.94 (d, J=8.8 Hz, 2H); ¹³C NMR (100 MHz, DMSO-d₆): δ 159.8, 155.1, 128.5, 116.0, 115.2; HRMS-ESI m/z calculated for C₇H₆N₄O [M+H]⁺ 163.0614, found 163.0613

3′-(Tetrazol-5-yl)-biphenyl-4-ol (13a): ¹H NMR (400 MHz, CD₃OD): δ 8.04 (d, J=8.8 Hz, 2H); 7.76 (d, J=8.0 Hz, 2H); 7.25 (t, J=8.0 Hz, 1H); 7.12-7.09 (m, 1H); 7.06-7.05 (m, 1H); 6.79-6.76 (m, 1H). ¹³C NMR (100 MHz, CD₃OD): δ 159.2, 145.6, 142.4, 131.1, 128.9, 128.7, 124.2, 119.3, 116.2, 114.8. HRMS-ESI m/z calculated for C₁₃H₁₁N₄O [M+H]⁺: 239.0927, found 239.0937.

4′-(Tetrazol-5-yl)-biphenyl-4-ol (14a): ¹H NMR (400 MHz, CD₃OD): δ 7.97 (d, J=8.8 Hz, 2H); 7.70 (d, J=8.4 Hz, 2H), 7.48 (d, J=8.8 Hz, 2H); 6.82 (d, J=8.8 Hz, 2H). ¹³C NMR (100 MHz, MeOD): δ 159.2, 157.4, 145.5, 132.3, 129.2, 128.7, 123.2, 116.9. HRMS-ESI m/z calculated for C₁₃H₁₁N₄O [M+H]⁺: 239.0927, found 239.0933.

(S)-2-(Tetrazol-5-yl)pyrrolidine-1-carboxylic Acid Benzyl Ester (15a): [α]_{D} at 24° C.=−90.4 (CHCl₃); ¹H NMR (400 CDCl₃ MHz,): δ 7.36-7.20 (5H, m), 5.22-5.10 (m, 3H), 3.53-3.50 (m, 2H), 2.99-2.93 (m. 1H), 2.36-2.28 (m, 1H), 2.20-2.05 (m, 3H); ¹³C NMR (100 MHz, CDCl₃): δ 156.8, 156.7, 135.8, 128.6, 128.4, 127.9, 68.0, 51.4, 47.2, 28.5, 24.7; HRMS-ESI m/z calculated for C₁₃H₁₅N₅O₂ [M+H]⁺: 274.1299, found 274.1308.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
reacting in a flow reactor an azide source and a compound of formula (I):
R¹—C≡N (I)
under conditions suitable for forming a compound of formula (II): 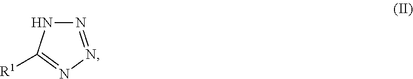
wherein R¹ is alkyl, aryl, heteroalkyl, or heteroaryl, each optionally substituted.

2. A method, comprising:
reacting an azide source and a compound of formula (I):
R¹—C≡N (I),
under conditions suitable for forming a compound of formula (II): 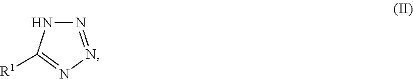
wherein the conditions are selected such that the percent conversion of compound (I) to compound (II) is greater than about 90% in a period of less than 1 hour and in the absence of microwave irradiation, and
wherein R¹ is alkyl, aryl, heteroalkyl, or heteroaryl, each optionally substituted.

3. A method, comprising:
reacting an azide source and a compound of formula (I):
R¹—C≡N (I),
under conditions suitable for forming a compound of formula (II): 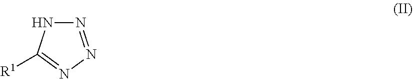
wherein the conditions comprise reacting at a temperature between about 150° C. and about 220° C. and in a solution comprising water and a polar aprotic solvent, wherein the ratio of water to polar aprotic solvent is between 1:9 and 9:1; and
wherein R¹ is alkyl, aryl, heteroalkyl, or heteroaryl, each optionally substituted.

4. The method of claim 1, wherein the reacting is carried out in the presence of a catalyst.

5. The method of claim 4, wherein the catalyst is ZrBr₂.

6. The method of claim 4, wherein the catalyst is provided in an amount of about 0.5 equiv, about 0.75 equiv, about 1 equiv, about 1.5 equiv, about 2 equiv, about 3 equiv, about 4 equiv, or about 5 equiv. of the compound of formula (I).

7. The method of claim 1, wherein the reacting is carried out in a flow reactor.

8. The method of claim 1, wherein the azide source is NaN₃.

9. The method of claim 1, wherein the ratio of the azide source to a compound about formula (I) is about 1:1, about 1.05:1, about 1.1:1, about 1.2:1, about 1.3:1, about 1.4:1, about 1.5:1, about 2:1, about 3:1, or about 4:1.

10. The method of claim 1, wherein the conditions comprise reacting at a temperature of about 150° C., about 160° C., about 170° C., about 180° C., about 190° C., about 200° C., about 210° C., about 220° C., or about 230° C.

11. The method of claim 1, wherein the conditions comprise reacting at a temperature between about 150° C. and about 220° C.

12. The method of claim 1, wherein the conditions comprise reacting in a solution comprising water and a polar aprotic solvent.

13. The method of claim 12, wherein the ratio of water to polar aprotic solvent is between about 1:9 and about 9:1.

14. The method of claim 1, wherein the reaction is carried out under conditions suitable for forming a compound of formula (II) in a percent conversion of greater than about 60%, greater than about 70%, greater than about 80%, greater than about 90%, greater than about 95%, greater than about 97%, or greater than about 99%, in a period of time of less than about 2 hours, less than about 1 hour, less than about 50 minutes, less than about 40 minutes, less than about 30 minutes, less than about 20 minutes, or less than about 10 minutes.

15. The method of claim 12, wherein the polar aprotic solvent is selected from the group consisting of N-methylpyrrolidone (NMP), dimethylformamide (DMF), dimethylacetamide (DMA), dimethylsulfoxide (DMSO), and combinations thereof.

16. The method of claim 1, wherein the azide source is selected from the group consisting of ammonium azide, lithium azide, sodium azide, potassium azide, rubidium azide, cesium azide, beryllium azide, magnesium azide, calcium azide, strontium azide, barium azide, and combinations thereof.

17. The method of claim 1, wherein R¹ is selected from the group consisting of 4-methoxy-phenyl; phenyl; naphthalene-2-yl; 4-benzaldehyde; 4-nitrophenyl; m-tolyl; 2-pyrazinyl; 2-pyridinyl; 6-methoxyquinoline; biphenyl-3-ol; p-tolyl; o-tolyl; 4-phenol; 3′-biphenyl-4-ol; 4′-biphenyl-4-ol; and (S)-2-pyrrolidine-1-carboxylic acid benzyl ester.

18. The method of claim 1, wherein the compound of formula (II) is a chiral compound.

19. The method of claim 1, wherein the compound of formula (II) is a chiral compound having an enantiomeric excess greater than about 95%.

20. The method of claim 1, wherein the reaction is carried out in the absence of a catalyst.
